(19) United States
(12) Patent Application Publication
(10) Pub. No.: US 2025/0064094 A1
(43) Pub. Date: Feb. 27, 2025

(54) COMPOSITIONS FOR FOOD COLORING AND ANTIOXIDANT SUPPLEMENTATION DERIVED FROM SORGHUM

(71) Applicant: REDLEAF BIOLOGICS, INC., Lexington, KY (US)

(72) Inventors: Chris Somerville, Tiburon, CA (US); Jordan Wood, Lexington, KY (US); Sean Voigt, Lexington, KY (US)

(73) Assignee: REDLEAF BIOLOGICS, INC., Lexington, KY (US)

(21) Appl. No.: 18/834,788

(22) PCT Filed: Feb. 2, 2023
(86) PCT No.: PCT/US2023/061860

§ 371 Date: Jul. 31, 2024

Related U.S. Application Data
(60) Provisional application No. 63/305,928, filed on Feb. 2, 2022.

(51) Int. Cl. A23L 5/43; A61K 31/352; A61K 36/899

(52) U.S. Cl. CPC A23L 5/43; A61K 31/352; A61K 36/899

(57) ABSTRACT

The present disclosure concerns compositions derived from extracts of a proprietary variety of Sorghum that provide excellent natural coloring antioxidant and functional ingredient properties. The compositions can be used in applications for coloration of a product, such as a food, a consumable, a garment, a fabric, a polymer, a beverage, a dietary supplement, or more. Due to the benign properties, the composition can be included in products where a red dye or red coloring is required, either alone or with other coloring agents to form a different color, hue, or shade.

### CROSS-REFERENCE TO RELATED APPLICATIONS

This application claims priority to U.S. Provisional Patent Application 63/305,928, filed Feb. 2, 2022, the content of which is hereby incorporated by reference in its entirety.

### FIELD

The present disclosure concerns compositions derived from extracts of a proprietary variety of Sorghum that provide excellent natural coloring, antioxidant and other functional ingredient properties.

### BACKGROUND

The identification of resources for degradable and safe compositions is increasingly desired to reduce risk to users as well as to eliminate complex manufacturing and toxic by-products. Natural resources can be particularly promising as the degradation and regeneration of the resource are straightforward and their safety for human application is understood. Accordingly, a need exists for generating useful products from natural resources.

### SUMMARY

A 1^{st} aspect of the present disclosure, either alone in combination with any other aspect herein, concerns a composition comprising luteolinidin, apigeninidin and a polymer.

A 2^{nd} aspect of the present disclosure, either alone in combination with any other aspect herein, concerns the composition of the 1^{st} aspect, wherein the polymer comprises luteloin and luteolin-7-glycoside.

A 3^{rd} aspect of the present disclosure, either alone in combination with any other aspect herein, concerns the composition of the 2^{nd} aspect, wherein the polymer further comprises luteolinidin and apigeninidin.

A 4^{th} aspect of the present disclosure, either alone in combination with any other aspect herein, concerns the composition of the 1^{st} aspect, further comprising luteolin monomers

A 5^{th} aspect of the present disclosure, either alone in combination with any other aspect herein, concerns the composition of the 1^{st} aspect, further comprising luteolin-7-glucoside monomers.

A 6^{th} aspect of the present disclosure, either alone in combination with any other aspect herein, concerns the composition of the 1^{st} aspect, further comprising quercetin monomers or ethers thereof.

A 7^{th} aspect of the present disclosure, either alone in combination with any other aspect herein, concerns the composition of the 1^{st} aspect, wherein the composition further comprises penduletin monomers.

An 8^{th} aspect of the present disclosure, either alone in combination with any other aspect herein, concerns the composition of the 1^{st} aspect, wherein the composition further comprises methoxyluteolinidin monomers.

A 9^{th} aspect of the present disclosure, either alone in combination with any other aspect herein, concerns the composition of the 1^{st} aspect, wherein the composition further comprises one or more isoschaftosides.

A 10^{th} aspect of the present disclosure, either alone in combination with any other aspect herein, concerns the composition of the 1^{st} aspect, wherein the composition further comprises ferulic acid.

An 11^{th} aspect of the present disclosure, either alone in combination with any other aspect herein, concerns the composition of the 1^{st} aspect, wherein the composition further comprises p-coumaric acid.

A 12^{th} aspect of the present disclosure, either alone in combination with any other aspect herein, concerns the composition of the 1^{st} aspect, wherein the composition is for use as a dietary supplement, a food colorant, a cosmetic ingredient, an antioxidant, or a dye.

A 13^{th} aspect of the present disclosure, either alone in combination with any other aspect herein, concerns a composition comprising luteolin monomers, luteolin-7-glucoside monomers and quercetin monomers or ethers thereof.

A 14^{th} aspect of the present disclosure, either alone in combination with any other aspect herein, concerns the composition of the 13^{th} aspect, further comprising apigeninidin monomers.

A 15^{th} aspect of the present disclosure, either alone in combination with any other aspect herein, concerns the composition of the 13^{th} aspect, further comprising luteolinidin monomers.

A 16^{th} aspect of the present disclosure, either alone in combination with any other aspect herein, concerns the composition of the 13^{th} aspect, further comprising polyphenol polymers, wherein the polyphenol polymers comprise luteolin and apigeninidin.

A 17^{th} aspect of the present disclosure, either alone in combination with any other aspect herein, concerns the composition of the 16^{th} aspect, wherein the polyphenol polymers further comprise a dimethyl ether of quercetin.

A 18^{th} aspect of the present disclosure, either alone in combination with any other aspect herein, concerns the composition of the 13^{th} aspect, wherein the composition further comprises penduletin monomers.

A 19^{th} aspect of the present disclosure, either alone in combination with any other aspect herein, concerns the composition of the 13^{th} aspect, wherein the composition further comprises apigeninidin monomers.

A 20^{th} aspect of the present disclosure, either alone in combination with any other aspect herein, concerns the composition of the 13^{th} aspect, wherein the composition further comprises methoxyluteolinidin monomers.

A 21^{st} aspect of the present disclosure, either alone in combination with any other aspect herein, concerns the composition of the 13^{th} aspect, wherein the composition further comprises one or more isoschaftosides.

A 22^{nd} aspect of the present disclosure, either alone in combination with any other aspect herein, concerns the composition of the 13^{th} aspect, wherein the composition further comprises ferulic acid.

A 23^{rd} aspect of the present disclosure, either alone in combination with any other aspect herein, concerns the composition of the 13^{th} aspect, wherein the composition further comprises p-coumaric acid.

A 24^{th} aspect of the present disclosure, either alone in combination with any other aspect herein, concerns the composition of the 13^{th} aspect, wherein the composition further comprises one or more polymers comprised of linked luteolinidin, luteolin, and quercetin or ethers thereof.

A 25^{th} aspect of the present disclosure, either alone in combination with any other aspect herein, concerns the composition of any preceding aspect, wherein the composition is derived from the leaf of Sorghum variety RedNatural.

A 26^{th} aspect of the present disclosure, either alone in combination with any other aspect herein, concerns a food coloring agent comprising the dried composition of the 1^{st} or 13^{th} aspect.

A 27^{th} aspect of the present disclosure, either alone in combination with any other aspect herein, concerns a food coloring agent comprising the composition of the 1^{st} or 13^{th} aspect and a diluent.

A 28^{th} aspect of the present disclosure, either alone in combination with any other aspect herein, concerns a food coloring agent comprising the composition of the 1^{st} or 13^{th} aspect and a preservative.

A 29^{th} aspect of the present disclosure, either alone in combination with any other aspect herein, concerns a dietary antioxidant comprising the dried composition of the 1^{st} or 13^{th} aspect.

A 30^{th} aspect of the present disclosure, either alone in combination with any other aspect herein, concerns a dietary antioxidant comprising the composition of the 1^{st} or 13^{th} aspect and a diluent.

A 31^{st} aspect of the present disclosure, either alone in combination with any other aspect herein, concerns a dietary antioxidant comprising the composition of the 1^{st} or 13^{th} aspect and a preservative.

A 32^{nd} aspect of the present disclosure, either alone in combination with any other aspect herein, concerns a method for preparing the composition of the 1^{st} or 13^{th} aspects, comprising solubilization of RedNatural Sorghum leaves and obtaining an extract therefrom.

A 33^{rd} aspect of the present disclosure, either alone in combination with any other aspect herein, concerns the method of the 28^{th} aspect, further comprising drying the extract.

A 34^{th} aspect of the present disclosure, either alone in combination with any other aspect herein, concerns the method of the 28^{th} aspect, further comprising obtaining a polymeric fraction within the extract, hydrolyzing the polymeric fraction to provide monomeric units; and, adding the monomeric units to the composition.

### BRIEF DESCRIPTION OF THE DRAWINGS

FIG. 1A shows absorbance at 475-550 nm of extract from RedNatural leaves.

FIG. 1B shows absorbance at 475-550 nm of extract from Sorghum Triple Trust Feed Milo leaves.

FIG. 2 shows an HPLC-PDA-ESI-MS/MS analysis of a standard leaf extract from the RedNatural variety, in which the absorbance of the eluate was measured at 260-700 nm in addition to the absorbance from 475-550 nm.

FIG. 3 shows an annotated HPLC chromatogram of the RedNatural leaf extract.

### DETAILED DESCRIPTION

The present disclosure concerns compositions of compounds obtained from extracts of Sorghum bicolor, particularly the Sorghum variety RedNatural. In some aspects, the extracts are obtained from the leaf of Sorghum variety RedNatural. Certain varieties of sorghum may accumulate deoxyanthocyanidins and other compounds in specialized tissues (e.g., leaf sheaths) or potentially at the site of certain infections. In some aspects, the compositions of the present disclosure are in an aqueous or hydrophilic solution. In other aspects, the compositions of the present disclosure may be adapted to a hydrophobic or organic solution. In some aspects, the compositions or parts thereof may be dissolved within a solution. In some aspects, the compositions or parts thereof may be suspended within a solution or immiscible within a solution. In further aspects, the compositions of the present disclosure may be in the form of a powder or other dried or semi-dried form, such as a paste. It is a further aspect that the compositions of the present disclosure can be incorporated into many products, such as foods for consumption, beverages, cosmetics, creams, tablets, emulsifiers, supplements, and many more human consumer goods.

It is an aspect of the present disclosure that some compositions of the present disclosure possess strong dying ability, such that the compositions of the present disclosure may be utilized as a dye and/or a coloring agent. As identified herein, the compositions of the present disclosure are extracted from Sorghum bicolor, particularly Sorghum variety RedNatural. As such, while the compositions are distinct from the plant, all of the compounds therein are non-toxic and/or safe for human application or consumption. It is a further aspect of the present disclosure that some compositions of the present disclosure possess strong antioxidant activity. In some aspects, the compositions can be used in applications for coloration of a product, such as a food, a consumable, a garment, a fabric, a polymer, a beverage, a dietary supplement, or more. Due to the benign properties, the composition can be included in products where a red dye or red coloring is required, cither alone or with other coloring agents to form a different color, hue, or shade.

In some aspects, the present disclosure concerns compositions of compounds obtained from Sorghum variety RedNatural extract. In some aspects, the compositions include compounds extracted from the leaf of Sorghum variety RedNatural. For the purposes of brevity, it should be understood that reference to RedNatural refers to the plant Sorghum variety RedNatural or a part thereof.

In some aspects, the compounds of the compositions are obtained from a series of steps of concentration and isolation from treated RedNatural leaves. It will be apparent, however, the RedNatural need not be the actual source of every compound of the compositions of the present disclosure. For example, as discussed below, it is in some aspects of the present disclosure that the compositions include luteolin-7-glucoside and/or luteolin. While such compounds may be obtained from RedNatural, the compositions of the present disclosure do not require it be extracted therefrom or that all compounds included in a composition be from RedNatural.

In some aspects of the present disclosure, the compositions of the present disclosure include one or more of luteolin, luteolin-7-glucoside, luteolinidin, apigeninidin, penduletin, methoxylutcolinidin (7-O-methyl and/or 5-O-methyl), dimethoxylutcolinidin, p-coumaric acid, ferulic acid, as well as any combination thereof. In some aspects, the compositions may include one or more deoxyanthocyanins selected from luteolinidin, apigeninidin, methoxyluteolinidin, and combinations thereof. In some aspects, the compositions may include one or more schaftoside isomers (including schaftoside 1 and schaftoside 2) and/or a quercetin dimethyl ether (DMEs) (including quercetin DME 1 and quercetin DME 2).

In some aspects, the compositions of the present disclosure may include a polymer. In some aspects, the polymer may include one or more of the polyphenols and/or flavonoids as set forth herein. In some aspects, the polymer may include cross-linked or covalently linked compounds that include at least one of luteolin, luteolin-7-glucoside, luteolinidin, apigeninidin, penduletin, methoxyluteolinidin, dimethoxyluteolinidin, p-coumaric acid, ferulic acid, one or more schaftoside isomers, one or more quercetin DMEs, or any combination thereof. In some aspects, the polymer may include at least one deoxyanthocyanin of luteolinidin, apigeninidin, methoxylutcolinidin, and/or dimethoxylutcolinidin. In some aspects, the polymer may further include at least one or more amino acids, peptides, proteins, carbohydrates, lignins, tannins, further phenols and/or flavonoids or any combination thereof. It will be appreciated that the polymers of the present disclosure need not be limited or restricted to polymers solely of polyphenol or flavonoid compounds.

In some aspects, the compositions may include one or more of the individual compounds of luteolin, lutcolin-7-glucoside, lutcolinidin, apigeninidin, penduletin, methoxylutcolinidin, dimethoxyluteolinidin, p-coumaric acid, ferulic acid, one or more schaftoside isomers (including schaftoside 1 and schaftoside 2), quercetin and/or one or more quercetin DMEs, or any combination thereof. In some aspects, the compositions may include a polymeric compound of cross-linked or covalently linked compounds that includes one or more or lutcolin, luteolin-7-glucoside, lutcolinidin, apigeninidin, pendulctin, methoxyluteolinidin, dimethoxyluteolinidin, p-coumaric acid, ferulic acid, one or more schaftoside isomers, quercetin DMEs, or any combination thereof. In some aspects, the compositions of the present disclosure may include one or more of luteolin, luteolin-7-glucoside, luteolinidin, apigeninidin, penduletin, methoxylutcolinidin, dimethoxyluteolinidin, p-coumaric acid, ferulic acid, one or more schaftoside isomers, quercetin and/or quercetin DMEs, or any combination thereof and a polymeric compound that includes one or more of luteolin, luteolin-7-glucoside, lutcolinidin, apigeninidin, penduletin, methoxyluteolinidin, dimethoxyluteolinidin, p-coumaric acid, ferulic acid, one or more schaftoside isomers, quercetin DMEs, or any combination thereof.

In some aspects, the present disclosure concerns a composition of a deoxyanthocyanin of lutcolinidin, apigeninidin, methoxyluteolinidin (and/or dimethoxyluteolinidin), lutcolinidin and apigeninidin, luteolinidin and methoxyluteolinidin (and/or dimethoxyluteolinidin), apigeninidin and methoxyluteolinidin (and/or dimethoxyluteolinidin), or luteolinidin, apigeninidin, and methoxyluteolinidin (and/or dimethoxyluteolinidin). In some aspects, the present disclosure concerns a composition that includes a polymer that at least in part includes a deoxyanthocyanin of luteolinidin, apigeninidin, methoxyluteolinidin (and/or dimethoxylutcolinidin), luteolinidin and apigeninidin, lutcolinidin and methoxyluteolinidin (and/or dimethoxylutcolinidin), apigeninidin and methoxyluteolinidin (and/or dimethoxyluteolinidin), or lutcolinidin, apigeninidin, and methoxyluteolinidin (and/or dimethoxyluteolinidin). In further aspects, the present disclosure concerns a composition of a deoxyanthocyanin of luteolinidin, apigeninidin, methoxyluteolinidin (and/or dimethoxylutcolinidin), lutcolinidin and apigeninidin, luteolinidin and methoxyluteolinidin (and/or dimethoxyluteolinidin), apigeninidin and methoxylutcolinidin (and/or dimethoxyluteolinidin), or lutcolinidin, apigeninidin, and methoxyluteolinidin (and/or dimethoxyluteolinidin) and a polymer that at least in part includes a deoxyanthocyanin of lutcolinidin, apigeninidin, methoxyluteolinidin (and/or dimethoxylutcolinidin), lutcolinidin and apigeninidin, luteolinidin and methoxyluteolinidin (and/or dimethoxyluteolinidin), apigeninidin and methoxylutcolinidin (and/or dimethoxyluteolinidin), or lutcolinidin, apigeninidin, and methoxylutcolinidin (and/or dimethoxylutcolinidin).

In some aspects, the composition may include between 0.1 to 70% weight by weight of the composition of at least one of luteolin, luteolin-7-glucoside, luteolinidin, apigeninidin, penduletin, methoxyluteolinidin, dimethoxyluteolinidin, p-coumaric acid, ferulic acid, one or more schaftoside isomers, quercetin and/or a quercetin DME, including about 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, and 69% w/w.

In some aspects, the compositions of the present disclosure contain one or more polyphenolic and/or flavonoid compounds as set forth herein. In some aspects, the polyphenolic and/or flavonoid content of the composition may be of from about 0.1 to about 50% by weight of the composition, including about 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, and 49% w/w.

In some aspects, the composition may include a polymer of between 0.1 to 30% weight by weight of the composition, wherein the polymer is cross-linked or covalently linked compounds that include at least in part one or more of luteolin, luteolin-7-glucoside, luteolinidin, apigeninidin, penduletin, methoxyluteolinidin, dimethoxyluteolinidin, p-coumaric acid, ferulic acid, one or more schaftoside isomers, quercetin and/or a quercetin DME, including about 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, and 29% w/w.

In some aspects, the composition may include from about 0.1 to about 20% w/w apigeninidin, including about 0.2, 0.3, 0.4, 0.5, 0.6, 0.7, 0.8, 0.9, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 30.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10, 11, 12, 13, 14, 15, 16, 17, 18, and 19% w/w.

In some aspects, the composition may include from about 0.1 to about 20% w/w luteolinidin, including about 0.2, 0.3, 0.4, 0.5, 0.6, 0.7, 0.8, 0.9, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 30.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10, 11, 12, 13, 14, 15, 16, 17, 18, and 19% w/w.

In some aspects, the composition may include from about 0.1 to about 20% w/w luteolin, including about 0.2, 0.3, 0.4, 0.5, 0.6, 0.7, 0.8, 0.9, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 30.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10, 11, 12, 13, 14, 15, 16, 17, 18, and 19% w/w.

In some aspects, the composition may include from about 0.1 to about 20% w/w methoxyluteolinidin and/or dimethoxyluteolinidin, including about 0.2, 0.3, 0.4, 0.5, 0.6, 0.7, 0.8, 0.9, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 30.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10, 11, 12, 13, 14, 15, 16, 17, 18, and 19% w/w.

In some aspects, the composition may include from about 0.1 to about 20% w/w luteolin-7-glycoside, including about 0.2, 0.3, 0.4, 0.5, 0.6, 0.7, 0.8, 0.9, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 30.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10, 11, 12, 13, 14, 15, 16, 17, 18, and 19% w/w.

In some aspects, the composition may include from about 0.1 to about 20% w/w penduletin, including about 0.2, 0.3, 0.4, 0.5, 0.6, 0.7, 0.8, 0.9, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 30.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10, 11, 12, 13, 14, 15, 16, 17, 18, and 19% w/w.

In some aspects, the composition may include from about 0.1 to about 20% w/w quercetin DME 1 and/or quercetin DME 2, including about 0.2, 0.3, 0.4, 0.5, 0.6, 0.7, 0.8, 0.9, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 30.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10, 11, 12, 13, 14, 15, 16, 17, 18, and 19% w/w.

In some aspects, the composition may include from about 0.1 to about 20% w/w schaftoside 1 and/or schaftoside 2, including about 0.2, 0.3, 0.4, 0.5, 0.6, 0.7, 0.8, 0.9, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 30.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10, 11, 12, 13, 14, 15, 16, 17, 18, and 19% w/w.

In some aspects, the composition may include from about 0.1 to about 20% w/w p-coumaric acid, including about 0.2, 0.3, 0.4, 0.5, 0.6, 0.7, 0.8, 0.9, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 30.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10, 11, 12, 13, 14, 15, 16, 17, 18, and 19% w/w.

In some aspects, the composition may include from about 0.1 to about 20% w/w ferulic acid, including about 0.2, 0.3, 0.4, 0.5, 0.6, 0.7, 0.8, 0.9, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 30.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10, 11, 12, 13, 14, 15, 16, 17, 18, and 19% w/w.

In some aspects, the compositions of the present disclosure may further include any additional component required to adapt the composition for an intended end-product. For example, the compositions may further include additional dyes to achieve a different hue. In some aspects, the compositions may include lipids, emulsifiers, surfactants, and/or creams to provide a topical composition.

In some aspects, the compositions of the present disclosure include luteolinidin, apigeninidin, and a polymer as set forth herein. In some aspects, the polymer may include at least in part luteolinidin and/or apigeninidin. In some aspects, the polymer may include at least in part luteolin and/or luteolin-7-glycoside. In some aspects, the polymer may include at least in part luteolin, luteolin-7-glycoside, luteolinidin, and apigeninidin.

In some aspects, the composition may include luteolinidin, apigeninidin, luteolin and the polymers as set forth herein. In some aspects, the composition may include luteolinidin, apigeninidin, luteolin luteolin-7-glycoside, and the polymers as set forth herein.

In some aspects, the compositions of the present disclosure may include luteolinidin, apigeninidin, quercetin and/or ethers thereof, and the polymers as set forth herein. In some aspects, the composition may include luteolinidin, apigeninidin, luteolin, luteolin-7-glycoside, quercetin and/or ethers thereof, and the polymers as set forth herein.

In some aspects, the compositions of the present disclosure may include luteolinidin, apigeninidin, penduletin and a polymer as set forth herein. In some aspects, the compositions may further include luteolin and/or luteolin-7-glycoside and/or quercetin and/or an ether thereof.

In some aspects, the compositions of the present disclosure may include luteolinidin, apigeninidin, methoxyluteolinidin (and/or dimethoxyluteolinidin) and a polymer as set forth herein. In some aspects, the compositions may further include luteolin and/or penduletin and/or luteolin-7-glycoside and/or quercetin and/or an ether thereof.

In some aspects, the compositions of the present disclosure may include luteolinidin, apigeninidin, one or more schaftosides, and a polymer as set forth herein. In some aspects, the compositions may further include luteolin and/or penduletin and/or methoxyluteolinidin (and/or dimethoxyluteolinidin) and/or luteolin-7-glycoside and/or quercetin and/or an ether thereof.

In some aspects, the compositions of the present disclosure may include luteolinidin, apigeninidin, ferulic acid, and a polymer as set forth herein. In some aspects, the compositions may further include luteolin and/or penduletin and/or methoxyluteolinidin (and/or dimethoxylutcolinidin) and/or luteolin-7-glycoside and/or one or more schaftosides and/or quercetin and/or an ether thereof.

In some aspects, the compositions of the present disclosure may include lutcolinidin, apigeninidin, p-coumaric acid, and a polymer as set forth herein. In some aspects, the compositions may further include luteolin and/or penduletin and/or ferulic acid and/or methoxyluteolinidin (and/or dimethoxyluteolinidin) and/or luteolin-7-glycoside and/or one or more schaftosides and/or quercetin and/or an ether thereof.

In some aspects, the compositions of the present disclosure may include luteolin, luteolin-7-glycoside, and quercetin and/or an ether thereof. In some aspects, the compositions of the present disclosure may include luteolin, luteolin-7-glycoside, quercetin and/or an ether thereof and a polymer as set forth herein.

In some aspects, the compositions of the present disclosure may include luteolin, luteolin-7-glycoside, apigeninidin, and quercetin and/or an ether thereof. In some aspects, the compositions of the present disclosure may include luteolin, luteolin-7-glycoside, apigeninidin, quercetin and/or an ether thereof and a polymer as set forth herein.

In some aspects, the compositions of the present disclosure may include luteolin, luteolin-7-glycoside, luteolinidin, and quercetin and/or an ether thereof. In some aspects, the compositions of the present disclosure may include luteolin, luteolin-7-glycoside, lutcolinidin, quercetin and/or an ether thereof and a polymer as set forth herein. In some aspects, the compositions of the present disclosure may include further include apigeninidin.

In some aspects, the compositions of the present disclosure may include luteolin, luteolin-7-glycoside, and quercetin and/or dimethyl ether thereof. In some aspects, the compositions of the present disclosure may include luteolin, luteolin-7-glycoside, quercetin and/or a dimethyl ether thereof and a polymer as set forth herein. In some aspects, the compositions of the present disclosure may include further include apigeninidin and/or luteolinidin.

In some aspects, the compositions of the present disclosure may include luteolin, luteolin-7-glycoside, penduletin, and quercetin and/or an ether thereof. In some aspects, the compositions of the present disclosure may include luteolin, luteolin-7-glycoside, penduletin, quercetin and/or an ether thereof and a polymer as set forth herein. In some aspects, the compositions of the present disclosure may include further include apigeninidin and/or lutcolinidin and/or a dimethyl quercetin ether.

In some aspects, the compositions of the present disclosure may include luteolin, luteolin-7-glycoside, methoxyluteolinidin (and/or dimethoxyluteolinidin), and quercetin and/or an ether thereof. In some aspects, the compositions of the present disclosure may include luteolin, luteolin-7-glycoside, methoxyluteolinidin (and/or dimethoxyluteolinidin), quercetin and/or an ether thereof and a polymer as set forth herein. In some aspects, the compositions of the present disclosure may include further include apigeninidin and/or luteolinidin and/or penduletin and/or a dimethyl quercetin ether.

In some aspects, the compositions of the present disclosure may include luteolin, luteolin-7-glycoside, one or more schaftosides, and quercetin and/or an ether thereof. In some aspects, the compositions of the present disclosure may include luteolin, luteolin-7-glycoside, one or more schaftosides, quercetin and/or an ether thereof and a polymer as set forth herein. In some aspects, the compositions of the present disclosure may include further include apigeninidin and/or lutcolinidin and/or penduletin and/or methoxyluteolinidin (and/or dimethoxyluteolinidin) and/or a dimethyl quercetin ether.

In some aspects, the compositions of the present disclosure may include luteolin, luteolin-7-glycoside, ferulic acid, and quercetin and/or an ether thereof. In some aspects, the compositions of the present disclosure may include luteolin, luteolin-7-glycoside, ferulic acid, quercetin and/or an ether thereof and a polymer as set forth herein. In some aspects, the compositions of the present disclosure may include further include apigeninidin and/or luteolinidin and/or penduletin and/or methoxyluteolinidin (and/or dimethoxyluteolinidin) and/or one or more schaftosides and/or a dimethyl quercetin ether.

In some aspects, the compositions of the present disclosure may include luteolin, luteolin-7-glycoside, p-coumaric acid, and quercetin and/or an ether thereof. In some aspects, the compositions of the present disclosure may include luteolin, luteolin-7-glycoside, p-coumaric acid, quercetin and/or an ether thereof and a polymer as set forth herein. In some aspects, the compositions of the present disclosure may include further include apigeninidin and/or luteolinidin and/or penduletin and/or methoxyluteolinidin (and/or dimethoxyluteolinidin) and/or ferulic acid and/or one or more schaftosides and/or a dimethyl quercetin ether.

In some aspects, the compositions of the present disclosure may include one or more phenolic and/or flavonoid compounds obtained from Sorghum variety RedNatural. In some aspects, the compounds may be obtained from the leaf of Sorghum variety RedNatural. In some aspects, the compounds are extracted by solubilization of compounds within the leaf, followed by a series of filtration and concentration steps to obtain solutions or slurries of the desired compounds. In some aspects, additional compounds can be removed by altering the pH and/or temperature, followed by filtration of the remaining compounds.

While the following describes analysis of compounds identified in RedNatural, it will be apparent that the compositions set forth herein can include further isolated compounds from RedNatural and also include the addition of further compounds, such as additional amino acids, peptides, vitamins, antioxidants, surfactants, polymers, emulsifiers, water, flavonoids and similar. In some aspects, the compositions may include a food coloring agernt and/or an antioxidant. Such may include a dried composition, as well as the presence of a preservative. Such may also include a diluent.

Also contemplated are methods of preparing the compositions as set forth herein. In aspects, such may include solubilization of RedNatural Sorghum leaves and obtaining an extract therefrom. Such may further include drying the extract. Such may include obtaining a polymeric fraction within the extract, hydrolyzing the polymeric fraction to provide monomeric units; and, adding the monomeric units to the composition.

### Examples

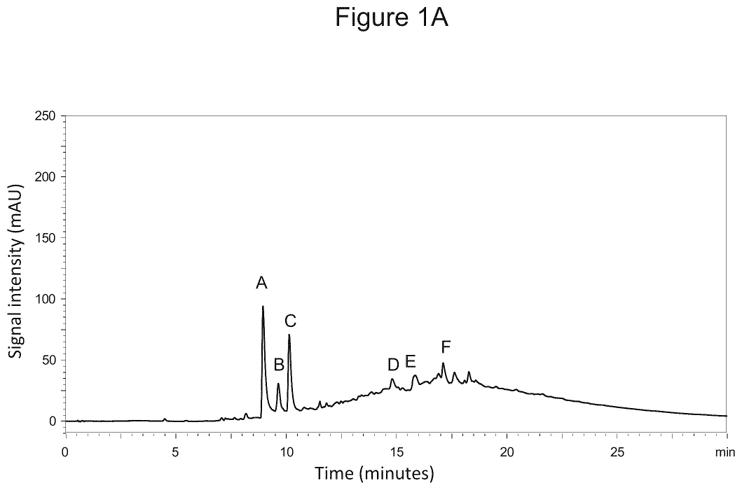

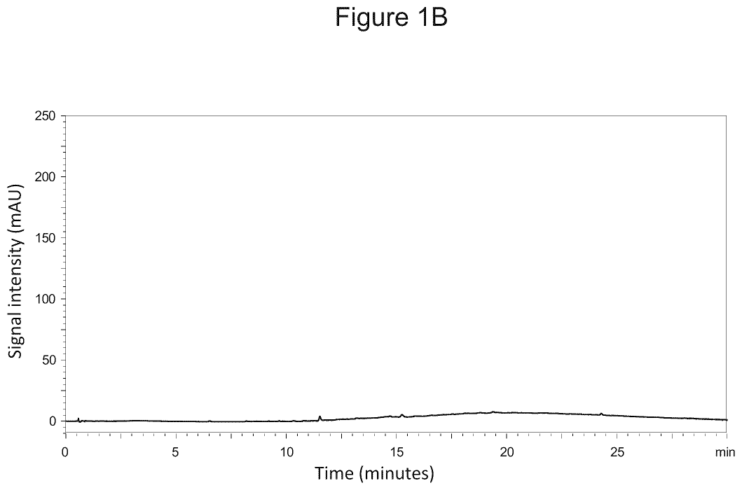

A concentrated solution of compounds obtained after solubilization and pH adjustment of RedNatural leaves was monitored at 475-550 nm and compared with extract from Sorghum variety Triple Trust Feed Milo. FIG. 1A shows the spectra obtained from RedNatural and FIG. 1 B shows that from the control (Triple Trust Feed Milo). RedNatural contains red-colored compounds that are not available in appreciable quantities from leaves of commonly used sorghum varieties.

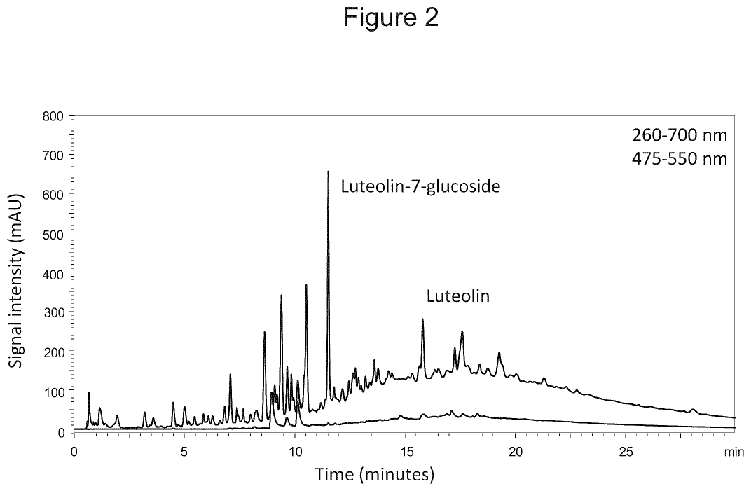

HPLC-PDA-ESI-MS/MS analysis was performed in which the absorbance of the eluate was measured at 260-700 nm in addition to the absorbance from 475-550 nm. The obtained data are set forth in FIG. 2. The identity of the various peaks was determined by mass spectrometry according to standard methods of analytical chemistry with the presence of significant quantities of luteolin and a glycoside being notable.

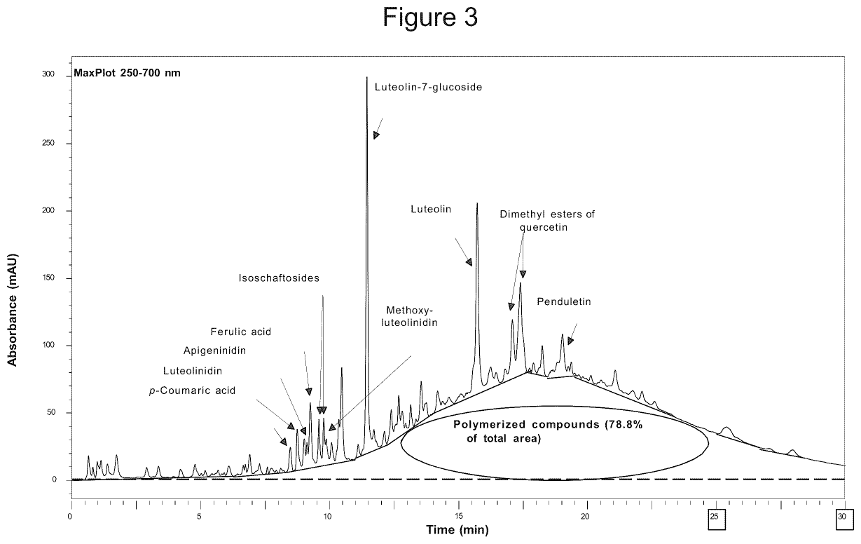

Next, three dried extracts from RedNatural leaves were evaluated for their total phenolic, flavonoid, and pigment content using spectrophotometric and chromatographic techniques. Samples belonged to three different production batches identified as B30, B31, and B34. Powders were dissolved in deionized water before spectrophotometric and chromatographic analyses. Composition was evaluated using ultra-high-performance liquid chromatography coupled with photodiode array detection and electrospray ionization with tandem mass spectroscopy (uHPLC-PDA-ESI-MS/MS). Extracts had a total phenolic content of 410.9±18.8 mg of gallic acid equivalents per gram of sample (˜41% weight). From that, flavonoids accounted for 144.9±4.0 mg of catechin equivalents per gram of sample (˜15%), and total pigment represented 86.3±1.6 mg of lutcolinidin equivalents per gram of sample (˜8%). The major compounds identified were p-coumaric acid, ferulic acid, two schaftosides isomers, luteolin-7-glucoside, luteolin, quercetin dimethyl ethers, penduletin, and deoxyanthocyanins (luteolinidin, apigeninidin, and methoxyluteolinidin). These compounds accounted for ˜45% of resolved peaks in the chromatogram. Additionally, analyses also revealed the presence of polymeric compounds evidenced by a bump in the chromatogram baseline, representing ˜79% of the total area under the PDA max plot. Acid hydrolyses of the powders resulted in a decrease of the polymeric compounds and an increase of the peak areas of the main phenolics previously identified, suggesting that the polymers are made at least in part of the same compounds found as free components in the extract. Acid hydrolyses of a polymer-rich fraction obtained using size-exclusion chromatography resulted in the release of luteolin, previously identified free as well as an additional compound tentatively identified as tyramine. Overall, monomeric compounds accounted for ˜21%, while polymeric compounds accounted for ˜79% of the uHPLC-PDA signal. The amount of variation in composition can be evaluated by the variance in the three measurements. FIG. 3 sets forth an annotated HPLC diagram of the leaf extract.

Various modifications of the present disclosure, in addition to those shown and described herein, will be apparent to those skilled in the art of the above description. Such modifications are also intended to fall within the scope of the appended claims.

It is appreciated that all reagents are obtainable by sources known in the art unless otherwise specified.

It is also to be understood that this disclosure is not limited to the specific aspects and methods described herein, as specific components and/or conditions may, of course, vary. Furthermore, the terminology used herein is used only for the purpose of describing particular aspects of the present disclosure and is not intended to be limiting in any way. It will be also understood that, although the terms “first,” “second,” “third” etc. may be used herein to describe various elements, components, regions, layers, and/or sections, these elements, components, regions, layers, and/or sections should not be limited by these terms. These terms are only used to distinguish one element, component, region, layer, or section from another element, component, region, layer, or section. Thus, “a first element,” “component,” “region,” “layer,” or “section” discussed below could be termed a second (or other) element, component, region, layer, or section without departing from the teachings herein. Similarly, as used herein, the singular forms “a,” “an,” and “the” are intended to include the plural forms, including “at least one,” unless the content clearly indicates otherwise. “Or” means “and/or.” As used herein, the term “and/or” includes any and all combinations of one or more of the associated listed items. It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof. The term “or a combination thereof” means a combination including at least one of the foregoing elements.

Unless otherwise defined, all terms (including technical and scientific terms) used herein have the same meaning as commonly understood by one of ordinary skill in the art to which this disclosure belongs. It will be further understood that terms such as those defined in commonly used dictionaries, should be interpreted as having a meaning that is consistent with their meaning in the context of the relevant art and the present disclosure, and will not be interpreted in an idealized or overly formal sense unless expressly so defined herein.

Reference is made in detail to exemplary compositions, aspects and methods of the present disclosure, which constitute the best modes of practicing the disclosure presently known to the inventors. The Figures are not necessarily to scale. However, it is to be understood that the disclosed aspects are merely exemplary of the disclosure that may be embodied in various and alternative forms. Therefore, specific details disclosed herein are not to be interpreted as limiting, but merely as a representative basis for any aspect of the disclosure and/or as a representative basis for teaching one skilled in the art to variously employ the present disclosure.

Patents, publications, and applications mentioned in the specification are indicative of the levels of those skilled in the art to which the disclosure pertains. These patents, publications, and applications are incorporated herein by reference to the same extent as if each individual patent, publication, or application was specifically and individually incorporated herein by reference.

The foregoing description is illustrative of particular aspects of the disclosure, but is not meant to be a limitation upon the practice thereof. The following claims, including all equivalents thereof, are intended to define the scope of the disclosure.

1. A composition comprising luteolinidin, apigeninidin and a polymer.

2. The composition of claim 1, wherein the polymer comprises luteloin and luteolin-7-glycoside.

3. The composition of claim 2, wherein the polymer further comprises luteolinidin and apigeninidin.

4. The composition of claim 1, further comprising luteolin monomers

5. The composition of claim 1, further comprising luteolin-7-glucoside monomers.

6. The composition of claim 1, further comprising quercetin monomers or ethers thereof.

7. The composition of claim 1, wherein the composition further comprises penduletin monomers.

8. The composition of claim 1, wherein the composition further comprises methoxyluteolinidin monomers.

9. The composition of claim 1, wherein the composition further comprises one or more isoschaftosides.

10. The composition of claim 1, wherein the composition further comprises ferulic acid.

11. The composition of claim 1, wherein the composition further comprises p-coumaric acid.

12. The composition of claim 1, wherein the composition is for use as a dietary supplement, a food colorant, a cosmetic ingredient, an antioxidant, or a dye.

13. A composition comprising luteolin monomers, luteolin-7-glucoside monomers and quercetin monomers or ethers thereof.

14. The composition of claim 13, further comprising apigeninidin monomers.

15. The composition of claim 13, further comprising luteolinidin monomers.

16. The composition of claim 13, further comprising polyphenol polymers, wherein the polyphenol polymers comprise luteolin and apigeninidin.

17. The composition of claim 16, wherein the polyphenol polymers further comprise a dimethyl ether of quercetin.

18. The composition of claim 13, wherein the composition further comprises penduletin monomers.

19. The composition of claim 13, wherein the composition further comprises apigeninidin monomers.

20. The composition of claim 13, wherein the composition further comprises methoxyluteolinidin monomers.

21. The composition of claim 13, wherein the composition further comprises one or more isoschaftosides.

22. The composition of claim 13, wherein the composition further comprises ferulic acid.

23. The composition of claim 13, wherein the composition further comprises p-coumaric acid.

24. The composition of claim 13, wherein the composition further comprises one or more polymers comprised of linked luteolinidin, luteolin, and quercetin or ethers thereof.

25. The composition of any preceding claim, wherein the composition is derived from the leaf of Sorghum variety RedNatural.

26. A food coloring agent comprising the dried composition of claim 1 or 13.

27. A food coloring agent comprising the composition of claim 1 or 13 and a diluent.

28. A food coloring agent comprising the composition of claim 1 or 13 and a preservative.

29. A dietary antioxidant comprising the dried composition of claim 1 or 13.

30. A dietary antioxidant comprising the composition of claim 1 or 13 and a diluent.

31. A dietary antioxidant comprising the composition of claim 1 or 13 and a preservative.

32. A method for preparing the composition of claim 1 or 13, comprising solubilization of RedNatural Sorghum leaves and obtaining an extract therefrom.

33. The method of claim 28, further comprising drying the extract.

34. The method of claim 28, further comprising obtaining a polymeric fraction within the extract, hydrolyzing the polymeric fraction to provide monomeric units; and, adding the monomeric units to the composition.
